(19) United States
(12) Patent Application Publication
(10) Pub. No.: US 2025/0054616 A1
(43) Pub. Date: Feb. 13, 2025

(54) MEDICAL PROCEDURE SCHEDULING SYSTEM

(71) Applicant: Medical Security Card Company, LLC, Tucson, AZ (US)

(72) Inventors: Jon Olaf JENSEN, New Canaan, CT (US); Marcus Nicholas SREDZINSKI, Tucson, AZ (US); Donna Marie ERCOLANO, San Diego, CA (US)

(21) Appl. No.: 18/799,469

(22) Filed: Aug. 9, 2024

Related U.S. Application Data
(60) Provisional application No. 63/532,239, filed on Aug. 11, 2023.

(51) Int. Cl. G16H 40/20; G16H 10/60; G16H 40/40

(52) U.S. Cl. CPC G16H 40/20; G16H 10/60; G16H 40/40

(57) ABSTRACT

A user system is configured to receive first information corresponding to a medical procedure and obtain profile information corresponding to the medical procedure, including at least a medical procedure name and a desired geographic region where the medical procedure will be performed. The user system is further configured to generate a plurality of queries about the medical procedure and send each of the plurality of queries to one of a plurality of medical procedure providers. The user system receives responses to each of the queries and generates an ordered list of the response, where the list is ordered by proximity to the desired geographic region or a cash price indicator for the medical procedure.

### RELATED APPLICATION

The present application claims priority to U.S. patent application No. 63/532,239 filed 11 Aug. 2023, which is incorporated herein by reference in its entirety.

### BACKGROUND

### Field of the Invention

The present disclosure is generally related to medical procedure scheduling systems and more particularly related to efficient scheduling of medical procedures for multiple doctors and multiple patients and multiple procedures.

### Related Art

Conventional medical procedure scheduling systems are cumbersome and ineffective at scheduling, leaving gaps in medical service provider schedules. Additionally, medical service providers in locations near to where a patient works are unable to offer their services to patients in need of those services because the patients in need are not aware of the medical service providers and the medical service providers are not aware of the patients in need. What is needed is a system and method that overcomes these significant problems found in the conventional commercial transactions as described above.

### SUMMARY

Accordingly, described herein are solutions to the problems in the conventional commercial transactions described above. In some aspects, the techniques described herein relate to a system for scheduling medical procedures, the system including: a user system configured to receive first information corresponding to a medical procedure and obtain profile information corresponding to the medical procedure and detailed information corresponding to the medical procedure, the profile information including a plurality of first data elements including at least one of a medical procedure name and a medical procedure code, the detailed information including at least a geographic region, wherein the user system is further configured to, generate a plurality of queries, each query including at least a portion of the profile information and at least the geographic region, send each of the plurality of queries to at least one of a plurality of medical procedure providers, each of the plurality of queries including at least a portion of the profile information and at least the geographic region, and receive a response to each of the plurality of queries, each response including second information including a plurality of second data elements including at least a cash price indicator for the medical procedure and a location where the medical procedure is performed; and a referral system configured to generate a report including one or more of the responses to pluralities of queries, wherein the report includes at least one of the first data elements and one of the second data elements, and wherein the referral system is configured to deliver the report via a user interface of the user system.

In some aspects, the techniques described herein relate to a system, wherein the medical procedure is one of a blood test, colonoscopy, broken bone setting, radiological exam, surgical procedures, a CT scan, and an elective medical procedure.

In some aspects, the techniques described herein relate to a system, wherein each of the plurality of queries includes a patient identifier and the detailed information further includes a medical history corresponding to the identified patient.

In some aspects, the techniques described herein relate to a system, wherein the report includes an ordered list sorted according to a proximity to the geographic region of the location where the medical procedure is performed.

In some aspects, the techniques described herein relate to a system, wherein the report includes an ordered list sorted according to the cash price indicator.

In some aspects, the techniques described herein relate to an apparatus for scheduling medical procedures, the apparatus including: a communication system configured for data communication via a data communication network; a non-transitory computer readable medium configured to store executable programmed modules; a user interface configured to receive first information corresponding to a medical procedure and present second information including at least a cash price indicator for the medical procedure and a location where the medical procedure is performed; and a processor communicatively coupled with the non-transitory computer readable medium and configured to execute programmed modules stored in the non-transitory computer readable medium, the processor further configured to control the communication system to carry out data communication over the data communication network and to control the user interface to receive the first information and provide the second information; wherein the processor is further configured to, control the user interface to obtain profile information corresponding to the medical procedure and detailed information corresponding to the medical procedure, the profile information including a plurality of first data elements including at least one of a medical procedure name and a medical procedure code, the detailed information including at least a geographic region; generate a plurality of queries, each query including at least a portion of the profile information and at least the geographic region, send each of the plurality of queries to at least one of a plurality of medical procedure providers, receive a response to each of the plurality of queries, each response including second information including a plurality of second data elements including at least a cash price indicator for the medical procedure and a location where the medical procedure is performed; and control the user interface to present an ordered list of responses to each of the plurality of queries.

In some aspects, the techniques described herein relate to an apparatus, wherein the medical procedure is one of a blood test, colonoscopy, broken bone setting, radiological exam, surgical procedures, a CT scan, and an elective medical procedure.

In some aspects, the techniques described herein relate to an apparatus, wherein each of the plurality of queries includes a patient identifier and the detailed information further includes a medical history corresponding to the identified patient.

In some aspects, the techniques described herein relate to an apparatus, wherein the ordered list is sorted according to a proximity to the geographic region of the location where the medical procedure is performed.

In some aspects, the techniques described herein relate to an apparatus, wherein the ordered list is sorted according to the cash price indicator.

In some aspects, the techniques described herein relate to a computer implemented method for scheduling medical procedures, where one or more processors are programmed to perform steps including: control a user interface to obtain profile information corresponding to a medical procedure and detailed information corresponding to the medical procedure, the profile information including a plurality of first data elements including at least one of a medical procedure name and a medical procedure code, the detailed information including at least a geographic region; generate a plurality of queries, each query including at least a portion of the profile information and at least the geographic region, send each of the plurality of queries to at least one of a plurality of medical procedure providers, receive a response to each of the plurality of queries, each response including second information including a plurality of second data elements including at least a cash price indicator for the medical procedure and a location where the medical procedure is performed; and control the user interface to present an ordered list of responses to each of the plurality of queries.

In some aspects, the techniques described herein relate to a method, wherein the medical procedure is one of a blood test, colonoscopy, broken bone setting, radiological exam, surgical procedures, a CT scan, and an elective medical procedure.

In some aspects, the techniques described herein relate to a method, wherein each of the plurality of queries includes a patient identifier and the detailed information further includes a medical history corresponding to the identified patient.

In some aspects, the techniques described herein relate to a method, wherein the ordered list is sorted according to a proximity to the geographic region of the location where the medical procedure is performed.

In some aspects, the techniques described herein relate to a method, wherein the ordered list is sorted according to the cash price indicator.

In some aspects, the techniques described herein relate to a non-transitory computer readable medium having stored thereon one or more sequences of instructions for causing one or more processors to perform steps for scheduling medical procedures, the steps including: control a user interface to obtain profile information corresponding to a medical procedure and detailed information corresponding to the medical procedure, the profile information including a plurality of first data elements including at least one of a medical procedure name and a medical procedure code, the detailed information including at least a geographic region; generate a plurality of queries, each query including at least a portion of the profile information and at least the geographic region, send each of the plurality of queries to at least one of a plurality of medical procedure providers, receive a response to each of the plurality of queries, each response including second information including a plurality of second data elements including at least a cash price indicator for the medical procedure and a location where the medical procedure is performed; and control the user interface to present an ordered list of responses to each of the plurality of queries.

In some aspects, the techniques described herein relate to a medium, wherein the medical procedure is one of a blood test, colonoscopy, broken bone setting, radiological exam, surgical procedures, a CT scan, and an elective medical procedure.

In some aspects, the techniques described herein relate to a medium, wherein each of the plurality of queries includes a patient identifier and the detailed information further includes a medical history corresponding to the identified patient.

In some aspects, the techniques described herein relate to a medium, wherein the ordered list is sorted according to a proximity to the geographic region of the location where the medical procedure is performed.

In some aspects, the techniques described herein relate to a medium, wherein the ordered list is sorted according to the cash price indicator.

In some aspects, the techniques described herein relate to a system including at least one processor communicatively coupled with at least one non-transitory computer readable medium, wherein the at least one processor is programmed to: control a user interface to obtain profile information corresponding to a medical procedure and detailed information corresponding to the medical procedure, the profile information including a plurality of first data elements including at least one of a medical procedure name and a medical procedure code, the detailed information including at least a geographic region; generate a plurality of queries, each query including at least a portion of the profile information and at least the geographic region, send each of the plurality of queries to at least one of a plurality of medical procedure providers, receive a response to each of the plurality of queries, each response including second information including a plurality of second data elements including at least a cash price indicator for the medical procedure and a location where the medical procedure is performed; and control the user interface to present an ordered list of responses to each of the plurality of queries.

In some aspects, the techniques described herein relate to a system, wherein the medical procedure is one of a blood test, colonoscopy, broken bone setting, radiological exam, surgical procedures, a CT scan, and an elective medical procedure.

In some aspects, the techniques described herein relate to a system, wherein each of the plurality of queries includes a patient identifier and the detailed information further includes a medical history corresponding to the identified patient.

In some aspects, the techniques described herein relate to a system, wherein the ordered list is sorted according to a proximity to the geographic region of the location where the medical procedure is performed.

In some aspects, the techniques described herein relate to a system, wherein the ordered list is sorted according to the cash price indicator.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 illustrates an example infrastructure, in which one or more of the processes described herein may be implemented, according to an embodiment;

FIG. 2 illustrates an example processing system, by which one or more of the processes described herein may be executed, according to an embodiment; and

FIG. 3 illustrates an example process for scheduling a medical procedure, according to an embodiment.

### DETAILED DESCRIPTION

Disclosed herein are systems, methods, and non-transitory computer-readable media for scheduling medical procedures. For example, one method disclosed herein allows for a scheduling apparatus to receive first information corresponding to a particular medical procedure and obtain profile information for the medical procedure. The profile information includes a medical procedure name and a geographic region in which the medical procedure is desired to be performed. Based on the profile information, a query corresponding to the medical procedure is sent to a plurality of medical procedure providers. A response to each of the queries is subsequently received, each response including second information including a proposed location to perform the medical procedure and a cash price indicator for the medical procedure. The responses can be compiled and presented in an ordered list based on the location or the price indicators to facilitate scheduling the medical procedure using the second information.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### 1. System Overview

### 1.1. Example Infrastructure

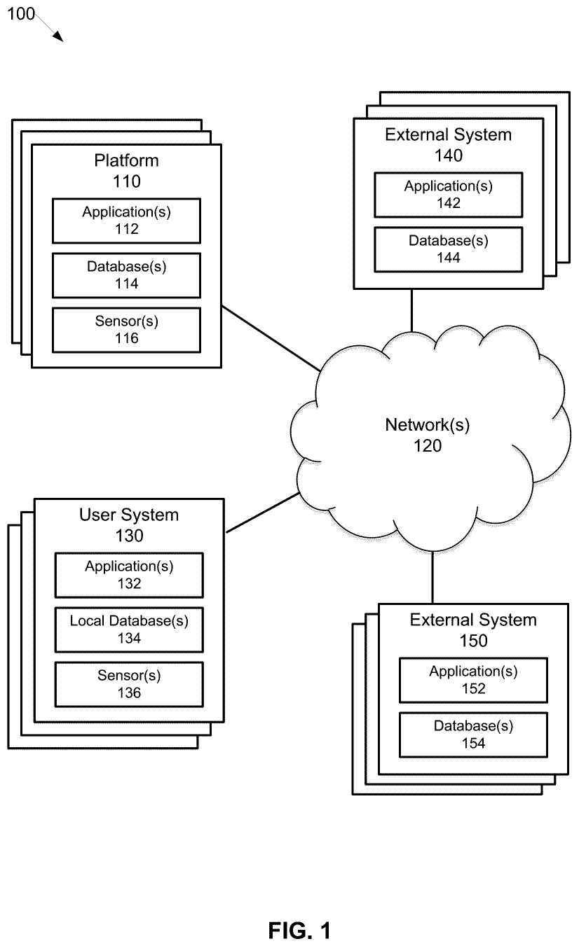

FIG. 1 illustrates an example infrastructure in which one or more of the disclosed processes may be implemented, according to an embodiment. The infrastructure may comprise a platform 110 (e.g., one or more servers or one or more point of sale (POS) devices) which hosts and/or executes one or more of the various functions, processes, methods, and/or software modules described herein. Platform 110 may comprise dedicated hardware and software, or may instead comprise cloud instances, which utilize shared resources of one or more servers. These POS devices, servers, and/or cloud instances may be collocated and/or geographically distributed. Platform 110 may also comprise or be communicatively connected to an application 112 and/or one or more databases 114. In addition, platform 110 may be communicatively connected to one or more user systems 130 via one or more networks 120. Platform 110 may also be communicatively connected to one or more external systems 140 and 150 (e.g., other platforms, websites, information repositories, databases, software-as-a-service platforms, etc.) via one or more networks 120.

In one aspect, the platform 110 is a server with an application 112 that works cooperatively with a plurality of user systems 130 and their respective applications 132. The applications 132 on the user systems 130 send queries to the application 112 on the platform 110 to obtain medical procedure profile information. External systems 140 and 150 may be affiliated with data repositories that maintain information about one or more patients, for example, medical information about a patient's medical history and prescriptions and other information about a patient that is helpful or necessary when scheduling a medical procedure for the patient. The user system 130 may send queries to the external systems 140 and 150 to obtained detailed information corresponding to the medical procedure or the patient.

External systems 140 and 150 may also be affiliated with one or more medical procedure service providers and may be configured to receive requests from one or more user systems 130 to schedule medical procedures. Such requests from user systems 130 may include, for example, the name of the medical procedure, a code corresponding to the medical procedure, a geographic region/location where the medical procedure is desired to be performed, and information about the corresponding patient. The external systems 140 and 150 are configured to respond to those requests with proposed dates, times, and locations for performing the requested medical procedure. The application 132 may also include a referral system configured to generate a report of the responses from the one or more medical procedure service providers. In one aspect, the referral system is configured to deliver the report via a user interface of the user system 130 and the report may be sorted according to a proximity of the proposed location to the desired location.

Network(s) 120 may comprise a personal area network, a mesh network, a local area network, a wide area network, the Internet, and any combination of wired or wireless networks. Networks(s) 120 may also include direct wired or wireless connections such as Bluetooth®. Platform 110 may communicate with user system(s) 130 through the network 120 using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via a private network such as an intranet or a direct communication link such as Bluetooth®. Furthermore, while only a few of each of the user systems 130, external systems 140, external systems 150, and platforms 110, are illustrated, it should be understood that the infrastructure may comprise any number of platforms, user systems, and external systems and any number of applications, databases, and sensors.

User system(s) 130 may comprise any type or types of computing devices capable of wired and/or wireless communication, including without limitation, desktop computers, laptop computers, tablet computers, smart phones or other mobile phones, servers, game consoles, head mounted displays, televisions, set-top boxes, electronic kiosks, point-of-sale terminals, Automated Teller Machines, and/or the like. In one aspect, user system 130 is a wireless communication device such as a smart phone that includes a sensor 136 such as a camera and the ability to communicate via a WIFI connection and also a cellular network connection.

External systems 140 and 150 may comprise web servers which host one or more websites and/or web services. In embodiments in which a website is provided, the website may comprise a graphical user interface, including, for example, one or more screens (e.g., webpages) generated in HyperText Markup Language (HTML) or other language. External systems 140 and 150 may also provide software-as-a-service capabilities and respond to queries that are received from, for example, platform 110 and/or user system 130. In doing so, external systems 140 and 150 may transmit or serve one or more screens of the graphical user interface in response to requests from platform 110 and/or user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to external systems 140 and 150 and the responses from external systems 140 and 150, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 144 and 154) that are locally and/or remotely accessible to external systems 140 and 150. External systems 140 and 150 may also respond to other requests from platform 110 and/or user system(s) 130.

Platform 110 may further comprise, be communicatively coupled with, or otherwise have access to one or more database(s) 114. For example, platform 110 may comprise one or more database servers which manage one or more databases 114. A user system 130 or application 112 executing on platform 110 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™, Access™, PostgreSQL™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, external systems 140 and 150 may receive requests from platform 110 and/or user system 130, and provide responses in extensible Markup Language (XML), JavaScript Object Notation (JSON), and/or any other suitable or desired format. In such embodiments, external systems 140 and 150 may provide an application programming interface (API) which defines the manner in which platform 110 and/or user system(s) 130 may interact with the web service. Thus, platform 110 and/or user system(s) 130, can define their own user interfaces, and rely on the web service to implement or otherwise provide the backend processes, methods, functionality, storage, and/or the like, described herein. For example, in such an embodiment, a client application 132 executing on one or more user system(s) 130 may interact with an application 142 or application 152 executing on external systems 140 and 150 to execute one or more or a portion of one or more of the various functions, processes, methods, and/or software modules described herein.

On the platform 110 and user system 130, application 112 and application 132 may be “thin,” in which case processing is primarily carried out server-side by applications 142 or 152 on external systems 140 and 150. A basic example of a thin client application 112 and/or 132 is a browser application, which simply requests, receives, and renders webpages at platform 110 and/or user system(s) 130, while server applications 142 and/or 152 on external systems 140 and 150 are responsible for generating the webpages and managing database functions. Alternatively, the client application may be “thick,” in which case processing is primarily carried out client-side by platform 110 or user system(s) 130. It should be understood that application 112 and/or application 132 may perform an amount of processing, relative to a server application 142 and/or 152 on external systems 140 and 150, at any point along this spectrum between “thin” and “thick,” depending on the design goals of the particular implementation. In any case, the application described herein, which may wholly reside on user system(s) 130 or be distributed between platform 110 and user system(s) 130 (e.g., in which case application 112 and application 132 both perform processing), can comprise one or more executable software modules that implement one or more of the processes, methods, or functions of the application described herein.

### 1.2. Example Processing Device

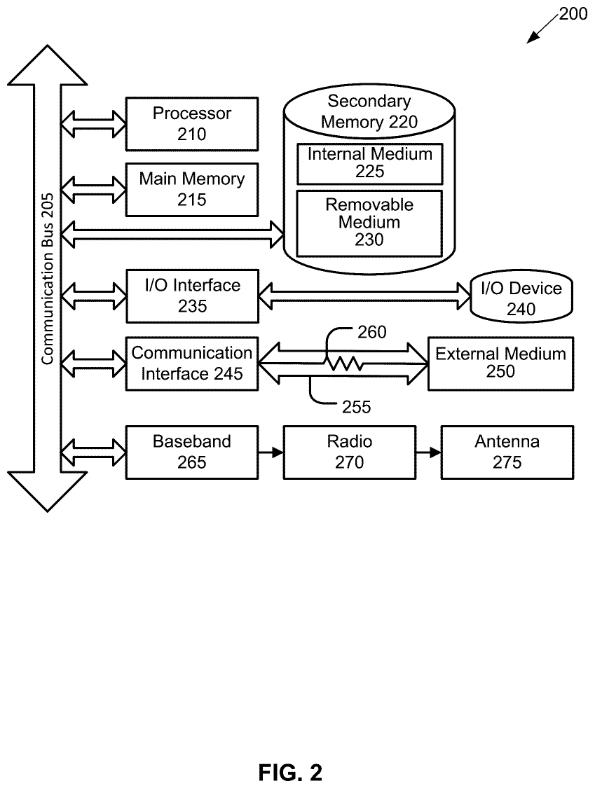

FIG. 2 is a block diagram illustrating an example wired or wireless system 200 that may be used in connection with various embodiments described herein. For example, system 200 may be used as or in conjunction with one or more of the functions, processes, or methods (e.g., to store and/or execute the application or one or more software modules of the application) described herein, and may represent components of platform 110, user system(s) 130, external system(s) 140, and/or other processing devices described herein. System 200 can be a smartphone device, a tablet computer, or any other processor-enabled device that is capable of wired or wireless data communication and preferable capable of data communication on a WIFI network and also on a cellular network. Other computer systems and/or architectures may be also used, as will be clear to those skilled in the art.

System 200 preferably includes one or more processors, such as processor 210. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 210. Examples of processors which may be used with system 200 include, without limitation, the Pentium® processor, Core i7® processor, and Xeon® processor, all of which are available from Intel Corporation of Santa Clara, California.

Processor 210 is preferably connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 preferably includes a main memory 215 and may also include a secondary memory 220. Main memory 215 provides storage of instructions and data for programs executing on processor 210, such as one or more of the functions and/or modules discussed herein. It should be understood that programs stored in the memory and executed by processor 210 may be written and/or compiled according to any suitable language, including without limitation C/C++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 220 may optionally include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

Secondary memory 220 is a non-transitory computer-readable medium having computer-executable code (e.g., disclosed software modules) and/or other data stored thereon. The computer software or data stored on secondary memory 220 is read into main memory 215 for execution by processor 210.

In alternative embodiments, secondary memory 220 may include other similar means for allowing computer programs or other data or instructions to be loaded into system 200. Such means may include, for example, a communication interface 245, which allows software and data to be transferred from external storage medium 250 to system 200. Examples of external storage medium 250 may include an external hard disk drive, an external optical drive, an external magneto-optical drive, and/or the like. Other examples of secondary memory 220 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, system 200 may include a communication interface 245. Communication interface 245 allows software and data to be transferred between system 200 and external devices (e.g. printers), networks, or other information sources. For example, computer software or executable code may be transferred to system 200 from a network server (e.g., platform 110) via communication interface 245. Examples of communication interface 245 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 245 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 245 are generally in the form of electrical communication signals 260. These signals 260 may be provided to communication interface 245 via a communication channel 255. In an embodiment, communication channel 255 may be a wired or wireless network (e.g., network(s) 120), or any variety of other communication links. Communication channel 255 carries signals 260 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed application, or software modules) is stored in main memory 215 and/or secondary memory 220. Computer programs can also be received via communication interface 245 and stored in main memory 215 and/or secondary memory 220. Such computer programs, when executed, enable system 200 to perform the various functions of the disclosed embodiments as described elsewhere herein.

In this description, the term “computer-readable medium” is used to refer to any non-transitory computer-readable storage media used to provide computer-executable code and/or other data to or within system 200. Examples of such media include main memory 215, secondary memory 220 (including internal memory 225, removable medium 230, and external storage medium 250), and any peripheral device communicatively coupled with communication interface 245 (including a network information server or other network device). These non-transitory computer-readable media are means for providing executable code, programming instructions, software, and/or other data to system 200.

In an embodiment that is implemented using software, the software may be stored on a computer-readable medium and loaded into system 200 by way of removable medium 230, I/O interface 235, or communication interface 245. In such an embodiment, the software is loaded into system 200 in the form of electrical communication signals 260. The software, when executed by processor 210, preferably causes processor 210 to perform one or more of the processes and functions described elsewhere herein.

In an embodiment, I/O interface 235 provides an interface between one or more components of system 200 and one or more input and/or output devices 240. Example input devices include, without limitation, sensors, cameras, microphones, keyboards, touch screens or other touch-sensitive devices, biometric sensing devices, computer mice, trackballs, pen-based pointing devices, and/or the like. Examples of output devices include, without limitation, other processing devices, speakers, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), head mounted displays (HMDs), and/or the like. In some cases, an input and output device 240 may be combined, such as in the case of a touch panel display (e.g., in a smartphone, tablet, or other mobile device).

In an embodiment, the I/O device 240 may be any type of external or integrated display and may include one or more discrete displays that in aggregate form the I/O device 240. The I/O device 240 may be capable of 2D or 3D presentation of visual information to a user of the system 200. In one embodiment, the I/O device 240 may be a virtual reality or augmented reality device in the form of HMD by the user so the user may visualize the presentation of information in 3D.

System 200 may also include optional wireless communication components that facilitate wireless communication over a voice network and/or a data network (e.g., in the case of user system 130). The wireless communication components comprise an antenna system 275, a radio system 270, and a baseband system 265. In system 200, radio frequency (RF) signals are transmitted and received over the air by antenna system 275 under the management of radio system 270.

In an embodiment, antenna system 275 may comprise one or more antennae and one or more multiplexors (not shown) that perform a switching function to provide antenna system 275 with transmit and receive signal paths. In the receive path, received RF signals can be coupled from a multiplexor to a low noise amplifier (not shown) that amplifies the received RF signal and sends the amplified signal to radio system 270.

In an alternative embodiment, radio system 270 may comprise one or more radios that are configured to communicate over various frequencies. In an embodiment, radio system 270 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 270 to baseband system 265.

If the received signal contains audio information, then baseband system 265 decodes the signal and converts it to an analog signal. Then the signal is amplified and sent to a speaker. Baseband system 265 also receives analog audio signals from a microphone. These analog audio signals are converted to digital signals and encoded by baseband system 265. Baseband system 265 also encodes the digital signals for transmission and generates a baseband transmit audio signal that is routed to the modulator portion of radio system 270. The modulator mixes the baseband transmit audio signal with an RF carrier signal, generating an RF transmit signal that is routed to antenna system 275 and may pass through a power amplifier (not shown). The power amplifier amplifies the RF transmit signal and routes it to antenna system 275, where the signal is switched to the antenna port for transmission.

Baseband system 265 is also communicatively coupled with processor 210, which may be a central processing unit (CPU). Processor 210 has access to data storage areas 215 and 220. Processor 210 is preferably configured to execute instructions (i.e., computer programs, such as the disclosed application, or software modules) that can be stored in main memory 215 or secondary memory 220. Computer programs can also be received from baseband processor 260 and stored in main memory 210 or in secondary memory 220, or executed upon receipt. Such computer programs, when executed, enable system 200 to perform the various functions of the disclosed embodiments.

### 1.3. Example Operation

In one example implementation of the presently described medical procedure scheduling system, a user system 130 receives the name of a particular medical procedure. The user system queries a platform 110 (e.g., a web server) for profile information about the medical procedure. The query is sent from the user system 130 to the platform 110 over a data communication network 120 such as the Internet. The platform 110 responds with the information about the medical procedure and the user system 130 receives the requested medical procedure profile information from the platform 110. In one aspect, the profile information about the medical procedure includes an identification of detailed information that is needed to schedule the medical procedure. For example, such detailed information may include current medications the patient is taking, patient prior medical procedure history, and patient allergies, just to name a few.

Once the user system 130 has received the medical procedure profile information, the user system uses the profile information to query one or more external systems 140 and 150 and/or request input via a user interface to obtain all of the detailed information that is needed to schedule the medical procedure. For example, detailed information needed to schedule the medical procedure may include the medical procedure name and alternative medical procedure names, one or more medical procedure codes corresponding to the medical procedure (e.g., a billing code), dates and times that the medical procedure is desired to be scheduled, and a desired geographic region where the medical procedure is to be performed.

Once the user system 130 has collected all of the detailed information needed to schedule the medical procedure, the user system 130 constructs and sends one or more queries to one or more medical procedure service providers (e.g., an external system 140 or 150 affiliated with such medical procedure service providers). The one or more queries includes the information necessary for the medical procedure service provider to schedule the appointment and also includes the user's desired geographic region/location for performing the medical procedure.

In response to the one or more queries, the user system 130 receives one or more responses with proposed schedules for performing the medical procedure at proposed dates/times and proposed locations. The user system 130 analyzes the one or more responses and organizes the response into an ordered list and presents the ordered list on a user interface. For example, the ordered list may be ordered based on a proximity to the desired geographic region/location. Alternatively, one or more of the one or more responses may include a cash price indicator for the medical procedure service provider to perform the medical procedure and the ordered list may be ordered based on the cash price indicator. Alternatively, the ordered list may be ordered based on both the proximity to the desired geographic region/location and the cash price indicator. In various aspects, the ordered list may be ordered based on one or more of: (a) geographic location/proximity to a desired geographic location; (b) cash price indicator; (c) insurance that is accepted; (d) government reimbursement programs that are accepted; and (e) consumer ratings, just to name a few.

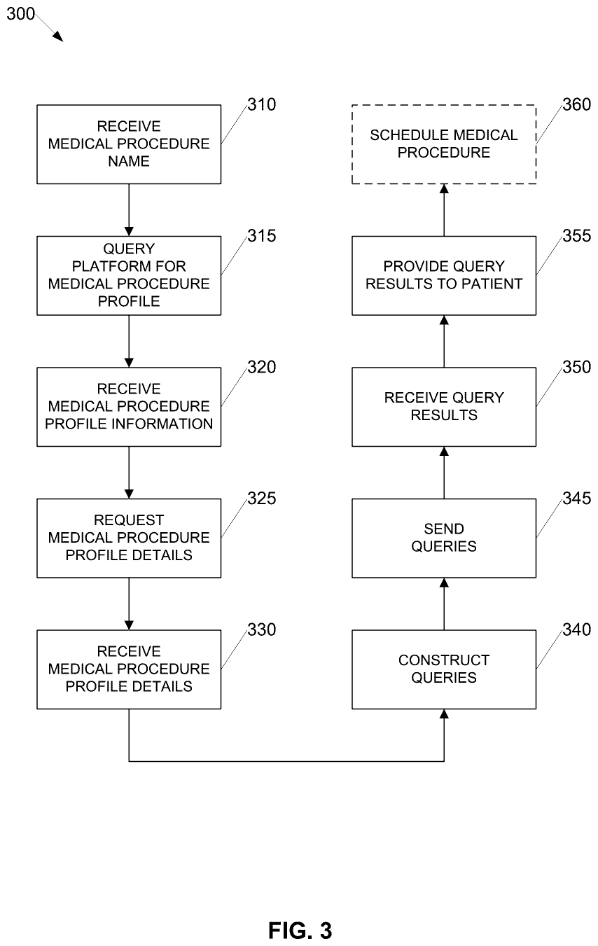

FIG. 3 illustrates an example process 300 for scheduling a medical procedure according to an embodiment. In one aspect, the process 300 may be carried out by the system described with respect to FIG. 1 in combination with one of more processing devices described with respect to FIG. 2.

Initially, at 310, the user system 130 receives a medical procedure name. The medical procedure name may be received as a text string comprising the name of the medical procedure. Alternatively, the medical procedure name may be received as a code such as a current procedural terminology (“CPT”) code or some other type of medical code. If a medical code is provided, the user system 130 is configured to lookup the code to translate the code into the actual medical procedure name or the user system 130 may instead subsequently use the code directly in a query.

Next, at 315 the user system 130 sends a query (e.g., a query to platform 110) to obtain profile information about the medical procedure. In response to the query, at 320 the user system 130 receives profile information for the medical procedure. The profile information may include a plurality of first data elements. The first data elements may include, for example, the medical procedure name, alternative medical procedure names, one or more medical procedure codes corresponding to the medical procedure, and other information about the medical procedure that may be necessary to provide to a medical procedure service provider to successfully schedule the medical procedure for a patient.

Next, at 325, the user system 130 requests medical procedure profile details. In one aspect, the user interface of the user system 130 may request information from a user of the user system 130 (e.g., a patient or medical professional or medical staff). In one aspect, the medical procedure profile details may in include a desired geographic region for performing the medical procedure, a desired date range and time of day for performing the medical procedure, current prescriptions that the patient is taking, and other detailed information that may be necessary or helpful in scheduling the particular medical procedure. Advantageously, gathering of medical procedure profile details results in the user system 130 obtaining all necessary information from the patient and/or the patient's medical record(s) (e.g., medical records may be hosted by one or more external systems 140 and 150) to successfully schedule the medical procedure for the patient with a medical procedure service provider. At 330, the user system 130 receives all of the requested medical procedure profile details for the identified medical procedure.

Next, at 340, the user system 130 generates a plurality of queries corresponding to the medical procedure and including at least a portion of the medical procedure profile details and at least a portion of the first data elements such as the medical procedure name and the medical procedure codes. The queries are generated in a format corresponding to a particular medical procedure service provider system. For example, each individual query may be formatted according to a defined application programming interface (API) or may be formatted according to a defined uniform resource locator (URL). Advantageously, the user system 130 may construct the one or more queries in parallel so that they may be used in parallel. In one aspect, each query includes a desired geographic region/location where the medical procedure is desired to be performed.

Next, at 345, the one or more queries are sent by the user system 130 to one or more medical procedure service providers (e.g., one or more external servers 140 and 150 affiliated with the one or more medical procedure service providers), where the queries may be processed in parallel. Such parallel processing of queries by the one or more medical procedure service providers significantly reduces the overall transaction time for scheduling the medical procedure with the ultimately selected medical procedure service provider. In one aspect, the user system 130 sends the queries to the medical procedure service providers via a data communication network 120, which may include the Internet.

Next, at 350 the user system 130 receives a response to each of the queries sent to each of the many medical procedure service providers. In one aspect, each response is received from an external system 140 or 150 via the data communication network 120 and each response includes a proposed date, time, and location for performing the medical procedure. In one aspect, each response also includes a cash price indicator representing the cost to be charged by the medical service provider for performing the medical procedure. The user system 130 analyzes the responses and constructs a summary of the responses to the queries. At 355, the summary of the responses is then presented by the user system 130 on a user interface of the user system 130. In one aspect, the summary of the responses may be presented as an ordered list based on the proximity to the desired geographic region/location from the query or based on the cash price indicator.

Next, at 360, in response to the patient selecting one of the medical procedure service providers, the user system 130 schedules the medical procedure for the patient with the selected medical procedure service provider.

In one aspect, constructing the queries in parallel and sending the queries to the many medical procedure service providers for processing in parallel and constructing a summary of the results as an ordered list based on the proximity to the geographic region or the cash price indicator significantly reduces the time for scheduling the medical procedure and improves the overall scheduling system across a plurality of medical procedure service providers. By significantly reducing the time require to schedule a plurality of medical procedures for a plurality of patients with a plurality of medical procedure service providers, the system improves the operation of the many medical procedure service providers disparate scheduling systems and significantly improves overall throughput of medical procedures provided by medical procedure service providers. The presently described system is particularly suitable for the scheduling of elective medical procedures.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. A system for scheduling medical procedures, the system comprising:
a user system configured to receive first information corresponding to a medical procedure and obtain profile information corresponding to the medical procedure and detailed information corresponding to the medical procedure, the profile information comprising a plurality of first data elements including at least one of a medical procedure name and a medical procedure code, the detailed information comprising at least a geographic region,
wherein the user system is further configured to,
a. generate a plurality of queries, each query comprising at least a portion of the profile information and at least the geographic region,
b. send each of the plurality of queries to at least one of a plurality of medical procedure providers, each of the plurality of queries comprising at least a portion of the profile information and at least the geographic region, and
c. receive a response to each of the plurality of queries, each response including second information comprising a plurality of second data elements including at least a cash price indicator for the medical procedure and a location where the medical procedure is performed; and
a referral system configured to generate a report comprising one or more of the responses to pluralities of queries, wherein the report includes at least one of the first data elements and one of the second data elements, and wherein the referral system is configured to deliver the report via a user interface of the user system.

2. The system of claim 1, wherein the medical procedure is one of a blood test, colonoscopy, broken bone setting, radiological exam, surgical procedures, a CT scan, and an elective medical procedure.

3. The system of claim 1, wherein each of the plurality of queries comprises a patient identifier and the detailed information further comprises a medical history corresponding to the identified patient.

4. The system of claim 1, wherein the report comprises an ordered list sorted according to a proximity to the geographic region of the location where the medical procedure is performed.

5. The system of claim 1, wherein the report comprises an ordered list sorted according to the cash price indicator.

6. An apparatus for scheduling medical procedures, the apparatus comprising:
a communication system configured for data communication via a data communication network;
a non-transitory computer readable medium configured to store executable programmed modules;
a user interface configured to receive first information corresponding to a medical procedure and present second information including at least a cash price indicator for the medical procedure and a location where the medical procedure is performed; and
a processor communicatively coupled with the non-transitory computer readable medium and configured to execute programmed modules stored in the non-transitory computer readable medium, the processor further configured to control the communication system to carry out data communication over the data communication network and to control the user interface to receive the first information and provide the second information;
wherein the processor is further configured to,
a. control the user interface to obtain profile information corresponding to the medical procedure and detailed information corresponding to the medical procedure, the profile information comprising a plurality of first data elements including at least one of a medical procedure name and a medical procedure code, the detailed information comprising at least a geographic region;
b. generate a plurality of queries, each query comprising at least a portion of the profile information and at least the geographic region,
c. send each of the plurality of queries to at least one of a plurality of medical procedure providers,
d. receive a response to each of the plurality of queries, each response including second information comprising a plurality of second data elements including at least a cash price indicator for the medical procedure and a location where the medical procedure is performed; and
e. control the user interface to present an ordered list of responses to each of the plurality of queries.

7. The apparatus of claim 6, wherein the medical procedure is one of a blood test, colonoscopy, broken bone setting, radiological exam, surgical procedures, a CT scan, and an elective medical procedure.

8. The apparatus of claim 6, wherein each of the plurality of queries comprises a patient identifier and the detailed information further comprises a medical history corresponding to the identified patient.

9. The apparatus of claim 6, wherein the ordered list is sorted according to a proximity to the geographic region of the location where the medical procedure is performed.

10. The apparatus of claim 6, wherein the ordered list is sorted according to the cash price indicator.

11. A computer implemented method for scheduling medical procedures, where one or more processors are programmed to perform steps comprising:
control a user interface to obtain profile information corresponding to a medical procedure and detailed information corresponding to the medical procedure, the profile information comprising a plurality of first data elements including at least one of a medical procedure name and a medical procedure code, the detailed information comprising at least a geographic region;
generate a plurality of queries, each query comprising at least a portion of the profile information and at least the geographic region,
send each of the plurality of queries to at least one of a plurality of medical procedure providers,
receive a response to each of the plurality of queries, each response including second information comprising a plurality of second data elements including at least a cash price indicator for the medical procedure and a location where the medical procedure is performed; and
control the user interface to present an ordered list of responses to each of the plurality of queries.

12. The method of claim 11, wherein the medical procedure is one of a blood test, colonoscopy, broken bone setting, radiological exam, surgical procedures, a CT scan, and an elective medical procedure.

13. The method of claim 11, wherein each of the plurality of queries comprises a patient identifier and the detailed information further comprises a medical history corresponding to the identified patient.

14. The method of claim 11, wherein the ordered list is sorted according to a proximity to the geographic region of the location where the medical procedure is performed.

15. The method of claim 11, wherein the ordered list is sorted according to the cash price indicator.

16. A non-transitory computer readable medium having stored thereon one or more sequences of instructions for causing one or more processors to perform steps for scheduling medical procedures, the steps comprising:
control a user interface to obtain profile information corresponding to a medical procedure and detailed information corresponding to the medical procedure, the profile information comprising a plurality of first data elements including at least one of a medical procedure name and a medical procedure code, the detailed information comprising at least a geographic region;
generate a plurality of queries, each query comprising at least a portion of the profile information and at least the geographic region,
send each of the plurality of queries to at least one of a plurality of medical procedure providers,
receive a response to each of the plurality of queries, each response including second information comprising a plurality of second data elements including at least a cash price indicator for the medical procedure and a location where the medical procedure is performed; and
control the user interface to present an ordered list of responses to each of the plurality of queries.

17. The medium of claim 16, wherein the medical procedure is one of a blood test, colonoscopy, broken bone setting, radiological exam, surgical procedures, a CT scan, and an elective medical procedure and wherein each of the plurality of queries comprises a patient identifier and the detailed information further comprises a medical history corresponding to the identified patient.

18. The medium of claim 16, wherein the ordered list is sorted according to the cash price indicator or a proximity to the geographic region of the location where the medical procedure is performed.

19. A system comprising at least one processor communicatively coupled with at least one non-transitory computer readable medium, wherein the at least one processor is programmed to:
control a user interface to obtain profile information corresponding to a medical procedure and detailed information corresponding to the medical procedure, the profile information comprising a plurality of first data elements including at least one of a medical procedure name and a medical procedure code, the detailed information comprising at least a geographic region;
generate a plurality of queries, each query comprising at least a portion of the profile information and at least the geographic region,
send each of the plurality of queries to at least one of a plurality of medical procedure providers,
receive a response to each of the plurality of queries, each response including second information comprising a plurality of second data elements including at least a cash price indicator for the medical procedure and a location where the medical procedure is performed; and
control the user interface to present an ordered list of responses to each of the plurality of queries.

20. The system of claim 19, wherein the medical procedure is one of a blood test, colonoscopy, broken bone setting, radiological exam, surgical procedures, a CT scan, and an elective medical procedure and wherein each of the plurality of queries comprises a patient identifier and the detailed information further comprises a medical history corresponding to the identified patient, and wherein the ordered list is sorted according to the cash price indicator or a proximity to the geographic region of the location where the medical procedure is performed.
